(19) United States
(12) Patent Application Publication
(10) Pub. No.: US 2010/0130620 A1
(43) Pub. Date: May 27, 2010

(54) NOVEL USE OF HYDROXYTYROSOL AND OLIVE EXTRACTS/CONCENTRATES CONTAINING IT

(76) Inventors: Jiankang Liu, Berkeley, CA (US); Wolfgang Schalch, Bottmingen (CH); Ying Wang-Schmidt, Stallikon (CH); Karin Wertz, Rheinfelden (DE)

Correspondence Address: NIXON & VANDERHYE, PC, 901 NORTH GLEBE ROAD, 11TH FLOOR, ARLINGTON, VA, 22203, US

(21) Appl. No.: 12/596,268

(22) PCT Filed: Apr. 18, 2007
(86) PCT No.: PCT/EP07/03405

§ 371 Date: Oct. 16, 2009

(51) Int. Cl. A61K 31/047; A61P 27/02

(52) U.S. Cl. 514/730

(57) ABSTRACT

The present invention is directed to the use of (a composition comprising) hydroxytyrosol for treating or preventing age-related macular degeneration in humans, for maintaining the eye health in animals (preferably in humans), especially in elderly animals (preferably elderly humans), for improving the vision of animals (preferably of humans), for maintaining the high resolution vision in animals (preferably in humans) and/or for maintaining the visual acuity in animals (preferably in humans), as well as for maintaining the visual performance in animals (preferably in humans) and/or the visual function in animals (preferably in humans).

The present invention is directed to the use of (a composition comprising) hydroxytyrosol for treating or preventing age-related macular degeneration in humans, for maintaining the eye health in animals (preferably in humans), especially in elderly animals (preferably elderly humans), for improving the vision of animals (preferably of humans), for maintaining the high resolution vision in animals (preferably in humans) and/or for maintaining the visual acuity in animals (preferably in humans), as well as for maintaining the visual performance in animals (preferably in humans) and/or the visual function in animals (preferably in humans).

Age-related macular degeneration (AMD) is, as the name implies an age-related degenerative condition of the macula. The macula represents the central part of the retina, which is essential to high resolution vision because there the density of photoreceptors (the light sensing cells) is at its maximum. Consequently, if the macula gets dysfunctional, visual tasks requiring high resolution such as recognizing faces or reading become progressively more difficult until, at the late stages of advanced AMD, they become impossible. In fact, this condition is the leading cause of blindness in the United States and other developed countries in the world.

Mostly beyond the age between 55 and 65, AMD begins with the build up of characteristic yellow deposits, called drusen, within and around the macular area. Most people with these early changes have still satisfactory vision but they are at risk to develop advanced AMD. This risk is considerably higher when the drusen are large and numerous and associated with disturbance in the pigmented cell layer (called the retinal pigment epithelium (RPE)) adjacent to the photoreceptors. Advanced AMD, which is responsible for profound vision loss, has two forms: dry and wet. Central geographic atrophy, the “dry” form of advanced AMD, causes these problems through loss of photoreceptors and cells supporting the photoreceptors in the central part of the eye. Currently, no treatment is available for this condition. Neovascular or exudative AMD, the “wet” form of advanced AMD, causes vision loss due to abnormal blood vessel growth (angiogenesis) beneath and into the macula. These newly formed blood vessels are imperfect and blood is leaking from them with the consequence that blood accumulates under the retina which leads to irreversible damage to the functional layers of the macula. Finally, if the condition is left untreated, vision is completely lost. While recently effective but very expensive treatment regimens for this neovascular (“wet”) form of AMD have become available, the ideal choice would still be to prevent this disease or at least to reduce the risk of this condition to develop.

In order to understand the etiology of AMD and its potential prevention or treatment it is important to realize that the photoreceptors are constantly exposed to oxidative damage in the environment of the retina which is characterized by the simultaneous presence of light and oxygen. As a consequence photoreceptors become damaged and dysfunctional and those “spent” photoreceptors have to be disposed of, while new photoreceptors have to be formed. The former task is accomplished by the RPE cells. These cells act under an enormous metabolic burden. It is estimated that during a period of about 10 days each single RPE cell has to phagocytose, digest and eliminate into the blood-flow about 50 photoreceptors. Thus during 60 years, more than 100'000 photoreceptors are to be processed by any single RPE cell. It is not surprising that during this intense metabolic activity digestion and elimination of spent photoreceptors is not always complete and cell debris is accumulating causing a progressive malfunctioning and eventual death of not only the RPE but also of the photoreceptor cells.

Logical targets for prevention of AMD, therefore, appear to be the following:

1: reduce oxidative damage by antioxidants;
2: reduce the amount of the most damaging blue light by yellow substances that specifically can absorb blue light such as lutein and zeaxanthin;
3: support the RPE cells that they are better able to cope with their extreme metabolic burden;
4: reduce the generation of new imperfect blood vessels by inhibiting angiogenesis.

The xanthophyll carotenoids lutein and zeaxanthin naturally accumulate in the central retina to the highest concentration seen everywhere in the human body. Therefore, and because of their blue light (which can cause damage to the retina) absorption and antioxidant characteristics, it is suggested that lutein and zeaxanthin can contribute to risk reduction of AMD. Supplementation with the dietary antioxidants vitamins C and E in combination with beta-carotene and zinc was already demonstrated to lower the risk of AMD progression. In monkeys fed a carotenoid-depleted diet for their entire life, the retinal pigment epithelium underneath the macula is defective in that it contains much less cells than in carotenoid-fed monkeys.

Moreover, the recently postulated importance of inflammation in the disease course of AMD is consistent with the expectation that cyclo-oxygenase (COX) inhibitors may be effective in AMD prevention, which was supported by the finding that specific COX inhibitors also inhibited VEGF (vascular endothelial growth factor). Consequently, also Aspirin was indicated to lower the risk of AMD. Another substance that has anti-angiogenic characteristics is genistein, the main ingredient of soy beans. Genistein may, therefore, have a relation to risk reduction of AMD, an idea which is supported by the observation that the prevalence of wet AMD among elderly in Asia is lower that that in age-matched Europeans.

The above mentioned substances are mainly suitable for reducing the risk of developing AMD. Very recently, antibodies against VEGF (vascular endothelial growth factor) for treatment of neovascular AMD have been made available. They have to be injected into the eye bulb, however, which is a risky and burdensome undertaking for the patient. Therefore, the medical need for AMD prevention and non-dangerous therapy for people with AMD or at risk of developing it, is still unsolved.

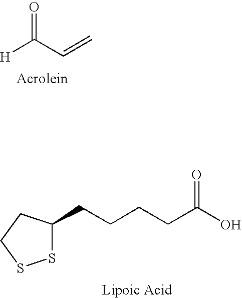

Recently the action of acrolein on cultured RPE cells has, to our knowledge for the first time, been described by Jia et al. in Invest Ophthalmol Vis Sci. 2007 January;48(1):339-348: Acrolein, a Toxicant in Cigarette Smoke, Causes Oxidative Damage and Mitochondrial Dysfunction in RPE Cells: Protection by (R)-α-Lipoic Acid. This publication documents that acrolein is a mitochondrial toxicant and that lipoic acid can reduce oxidative RPE damage leading to the conclusion that lipoic acid and compounds having a similar mechanism of action capable of treating or preventing AMD.

Surprisingly it has now been found, that hydroxytyrosol can also reduce oxidative RPE damage on cultured RPE cells that are exposed to the strong oxidant acrolein. One underlying mechanism of hydroxytyrosol's action may be via Nrf2, a key regulator of genes encoding antioxidant proteins and phase II enzymes, which neutralize free radicals and convert other toxic compounds in less reactive molecules. In this process, phase II enzymes, attach “neutralizing” elements to the unwanted substances making them easier for the body to excrete. Examples of phase II enzymes are Glutathione S-transferase, NAD(P)H:quinone oxidoreductase 1, UDP-glucuronosyltransferase, Gamma-glutamate cysteine ligase, and Hemeoxygenase-1 known to mediate enzymatic body detoxification and/or to exert antioxidant functions thereby protecting cells from toxic damage. Our data indicated that hydroxytyrosol increases Nrf2 protein level in APRE19 cells and subsequently, modulates GSH (reduced form of glutathione) and SOD (superoxide dismutase) level in a positive manner. In addition, hydroxytyrosol treatment protects cells from mitochondrial function decline and improves cell viability. Thus, hydroxytyrosol is capable of treating and/or preventing AMD in humans.

Oxidative stress and resulting damage of RPE cells are part of AMD pathogenesis. Acrolein causes oxidative stress in RPE cells. Hence, this cell culture model can serve to identify compounds which protect RPE cells from cell death after oxidative stress. One could assume that any antioxidant could protect RPE cells from acrolein-induced oxidative stress. We found that of an entire array of antioxidants tested, HT was the only one which could protect.

As mentioned in the introduction, the environment of the retina is characterized by the simultaneous presence of light and oxygen. This climate gives rise to the generation of numerous reactive oxygen species and highly reactive oxygen radicals, such as the hydroxyl- and superoxide radicals. Hydroxytyrosol, in the given examples has surprisingly been demonstrated to be able to ameliorate the toxic properties of acrolein.

Thus, hydroxytyrosol and derivatives thereof, as well as any olive juice/aqueous preparation/extract/concentrate containing it may be able of maintaining the visual performance and/or the visual function. Visual function is a prerequisite for visual performance, i.e. the performance of the visual task taking into consideration speed and accuracy. The visual task may encompass several abilities like reading of texts written in defined font sizes; visual acuity (to be able to see a sharp/focussed image) at different lighting conditions and for close as well as distant objects; the ability to discern details of a defined size in an image at defined lighting conditions (image resolution, contrast acuity (i.e. ability to see with a high image resolution at low light conditions (weak contrasts))), and the ability to accommodate fast between different lighting conditions.

### Hydroxytyrosol and Derivatives Thereof

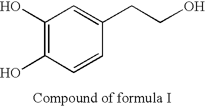

Hydroxytyrosol (compound of formula I; 3,4-dihydroxyphenyl ethanol) may be of synthetic origin or it may be obtained together with other water-soluble polyphenols such as tyrosol and oleuropein from extraction of olive leaves, olive fruits and vegetation water of olive oil production.

Examples of references that deal with the extraction of oleuropein and/or hydroxytyrosol from olive leaves are WO02/18310, US 2002/0198415, WO2004/005228, U.S. Pat. No. 6,416,808 and US 2002/0058078 which disclose a method for acidic hydrolysis of olive vegetation water for 2 to 12 months until at least 90% of the present oleuropein has been converted. A method of extraction of phenolic compounds from olives, olive pulps, olive oil and oil mill waste water is described by Usana Inc. patents U.S. Pat. No. 6,361,803 and WO01/45514 and in US 2002/0004077. EP-A 1 582 512 describes an extraction of hydroxytyrosol from olive leaves. A method for obtaining hydroxytyrosol and/or oleuropein from the vegetation water of de-pitted olives is disclosed in US 2004/0039066 A1 in paragraphs [0080]-[0091].

Derivatives may e.g. be esters. An example of a preferred ester of hydroxytyrosol is oleuropein.

The vegetation water may especially have been manufactured according to one of the processes disclosed in U.S. Pat. No. 6,416,808 (column 4, line 37 to column 7, line 27); WO 2004/005228; U.S. Pat. No. 6,936,287; US 2005-103 711; US 2003-108 651; US 2002-198 415; U.S. Pat. No. 6,165,475; JP 2001-252 054; JP 2000-319 161; WO 01/45514 (Usana); U.S. Pat. No. 6,358,542 (see especially column 4, line 1 to column 9, line 50 and examples 1-5 and 11-13); U.S. Pat. No. 6,361,803 (see especially column 3, line 64 to column 9, line 47 and examples 1-5 and 11-13); and WO 2006/084 658.

The vegetation water was preferably manufactured as disclosed in U.S. Pat. No. 6,416,808 (column 4, line 37 to column 7, line 27).

Instead of hydroxytyrosol also a vegetation water concentrate may be used; the use of hydroxytyrosol in a purity of at least 1.5 weight-%, preferably of at least 30 weight-%, more preferably of at least 50 weight-%, is however preferred.

An especially suitable vegetation water concentrate is e.g. “HIDROX® 6%”, commercially available from CreAgri, Hayward, USA. “HIDROX® 6%” contains 5 to 8 weight-% of proteins, 45 to 68 weight-% of carbohydrates, 17 to 30 weight-% of fat, 8 to 15 weight-% of ash and a minimum of 6 weight-% of water-soluble simple and polyphenols, based on the total weight of HIDROX® 6%.

“HIDROX® 2%” and “HIDROX® 9%”, both also commercially available from CreAgri, Hayward, USA, may also be used, as well as the following products commercially available from Glanbia and Indena (Milan, Italy): OLIVACTIV™ containing from 20 to 35 weight-% of hydroxytyrosol and from 4 to 6 weight-% of tyrosol; OLEASELECT™ having a total content of phenols of ≧30 weight-% (measured by UV) and an amount of hydroxytyrosol of ≧1.5 weight-% (measured by HPLC) and an amount of verbascoside of ≧5.0 weight-% (measured by HPLC) and OLIVE(OLEA)DRY, a powder containing from 22 to 24 g of hydroxytyrosol and from 5.0 to 6.5 g of tyrosol per kg.

Further suitable products are Prolivols, commercially available from Seppic, containing 35 weight-% of polyphenols, especially 20 mg hydroxytyrosol (per g of Prolivols) and 3 mg of tyrosol (per g of Prolivols); as well as Olive Braun Standard 500 (from obipektin): a powder containing from 1.0 to 2.2 g of hydroxytyrosol and from 0.2 to 0.7 g of tyrosol per kg; Olivex olive polyphenol liquid P10 (from Albert Isliker): a liquid containing from 2.0 to 3.5 g of hydroxytyrosol and from 0.2 to 1.0 g of tyrosol per kg; Olivex olive polyphenol (from Albert Isliker): a powder containing from 22 to 23 g of hydroxytyrosol and from 6.5 to 8.0 g of tyrosol per kg; and Olive Polyphenols NLT (from Lalilab Inc.) containing from 2.0 to 6 weight-% of hydroxytyrosol and from 0.7 to 1.1 weight-% of tyrosol.

In suitable commercially available vegetation water concentrates the amount of hydroxytyrosol varies in the range of from 1.0 to 220 g per kg of the total weight of the vegetation water concentrate. The amount of tyrosol preferably varies in the range of from 0.2 to 45 g per kg of the total weight of the vegetation water concentrate. The weight ratio of hydroxytyrosol to tyrosol is preferably between 100:10 and 100:40, most preferably between 100:18 and 100:35.

“Elderly humans” in the context of the present invention means humans at an age in the range of from 50 to 125, preferably at an age in the range of from 60 to 90.

“Treatment” in the context of the present invention is defined as oral application in order to stop or delay the progress of an eye disease.

“Prevention” in the context of the present invention is defined as intervention with the intention to reduce the risk of coming down with an eye disease.

“Maintaining the eye health” in the context of the present invention is defined as ensuring that the integrity of the eye, in particular of the retina with its different layers, remains fully or predominantly or partly functional.

“Improving the vision” in the context of the present invention means to generally improve visual performance, as measured by visual low resolution charts such as the ETDRS (Early Treatment Diabetic Retinopathy Study) Chart.

“Maintaining the high resolution vision” in the context of the present invention means to maintain in particular the reading ability as measured by reading charts.

“Maintaining the visual acuity” in the context of the present invention means to prevent a decline in visual acuity.

The visual acuity is the most common (but not the only) clinical measurement of visual function. Visual acuity is a quantitative measure of the ability to identify black symbols on a white background at a standardized distance as the size of the symbols is varied. The visual acuity represents the smallest size that just can be reliably identified on such a chart (often a so-called Snellen Chart).

Visual acuity is often expressed as a common fraction. Using the meter as the unit of measurement, this fractional visual acuity is expressed relative to 6/6. Having a visual acuity of 6/6 means that a letter size that normally should be identified from 6 meter is indeed identified from that distance. This is the best visual acuity and in the decimal system is represented a 1.0 meaning 100% visual acuity. Visual acuity of 6/60 means that from 6 meters a detail is seen which a person with normal vision would see at a distance of 60 meters, this represents 10% visual acuity. Visual acuity, however, can also be higher than 100%.

The daily dosage of hydroxytyrosol for humans (70 kg person) may vary from 5 to 500 mg, preferably from 15 to 100 mg.

The preferred dose of hydroxytyrosol varies from 0.28 to 1.9 mg/kg metabolic body weight for mammals, whereby

“metabolic body weight” [in kg]=(body weight [in kg])^{0.75}

for mammals. That means e.g. that for a human of 70 kg the preferred daily dose would vary between 6.77 and 45.98 mg, for a 20 kg dog the preferred daily dose would vary between 2.23 and 15.1 mg.

Animals in the context of the present invention encompass humans, pets (dogs, cats, birds (such as canaries, parrots, budgerigars, shell parakeets), farm animals, falcons and hawks, whereas humans are especially preferred.

The present invention is also directed to nutraceutical compositions comprising hydroxytyrosol for treating or preventing age-related macular degeneration in humans, for maintaining the eye health in animals (preferably in humans), especially in elderly animals (preferably elderly humans), for improving the vision of animals (preferably of humans), for maintaining the high resolution vision in animals (preferably in humans) and/or for maintaining the visual acuity in animals (preferably in humans), as well as for maintaining the visual performance in animals (preferably in humans) and/or the visual function in animals (preferably in humans).

The term nutraceutical composition as used herein include food product, foodstuff, dietary supplement, nutritional supplement or a supplement composition for a food product or a foodstuff, beverages (e.g. but not limited to sports beverages, functional waters, juices, smoothies; instant drinks), dairy products (e.g. but not limited to single shot yogurt drinks), nutritional bars, and spreads.

As used herein, the term food product refers to any food or feed suitable for consumption by humans or animals. The food product may be a prepared and packaged food (e.g., mayonnaise, salad dressing, bread, or cheese food) or an animal feed (e.g., extruded and pelleted animal feed, coarse mixed feed or pet food composition). As used herein, the term foodstuff refers to any substance fit for human or animal consumption. The term dietary supplement refers to a small amount of a compound for supplementation of a human or animal diet packaged in single or multiple dose units. Dietary supplements do not generally provide significant amounts of calories but may contain other micronutrients (e.g., vitamins or minerals). The term nutritional supplement refers to a composition comprising a dietary supplement in combination with a source of calories. In some embodiments, nutritional supplements are meal replacements or supplements (e.g., nutrient or energy bars or nutrient beverages or concentrates).

Food products or foodstuffs are for example beverages such as non-alcoholic and alcoholic drinks as well as liquid preparation to be added to drinking water and liquid food, non-alcoholic drinks are for instance soft drinks, sport drinks, fruit juices, such as for example orange juice, apple juice and grapefruit juice; vegetable juices such as tomato juice; lemonades, teas, near-water drinks and milk and other dairy drinks such as for example yoghurt drinks, and diet drinks.

In another embodiment food products or foodstuffs refer to solid or semi-solid foods comprising the composition according to the invention. These forms can include, but are not limited to baked goods such as cakes and cookies, puddings, dairy products, confections, snack foods, or frozen confections or novelties (e.g., ice cream, milk shakes), prepared frozen meals, candy, snack products (e.g., chips), liquid food such as soups, spreads, sauces, salad dressings, prepared meat products, cheese, yogurt and any other fat or oil containing foods, and food ingredients (e.g., wheat flour).

The term food products or foodstuffs also includes functional foods and prepared food products, the latter referring to any pre-packaged food approved for human consumption.

Animal feed including pet food compositions advantageously include food intended to supply necessary dietary requirements, as well as treats (e.g., dog biscuits) or other food supplements. The animal feed comprising the composition according to the invention may be in the form of a dry composition (for example, kibble), semi-moist composition, wet composition, or any mixture thereof. Alternatively or additionally, the animal feed is a supplement, such as a gravy, drinking water, yogurt, powder, suspension, chew, treat (e.g., biscuits) or any other delivery form.

Dietary supplements of the present invention may be delivered in any suitable format suitable for oral delivery. The ingredients of the dietary supplement of this invention are contained in acceptable excipients and/or carriers for oral consumption. The actual form of the carrier, and thus, the dietary supplement itself, is not critical. The carrier may be a liquid, gel, gelcap, capsule, powder, solid tablet (coated or non-coated), tea, or the like. The dietary supplement is preferably in the form of a tablet or capsule and most preferably in the form of a hard (shell) gelatin capsule. Suitable excipient and/or carriers include maltodextrin, calcium carbonate, dicalcium phosphate, tricalcium phosphate, microcrystalline cellulose, dextrose, rice flour, magnesium stearate, stearic acid, croscarmellose sodium, sodium starch glycolate, crospovidone, sucrose, vegetable gums, lactose, methylcellulose, povidone, carboxymethylcellulose, corn starch, and the like (including mixtures thereof). Preferred carriers include calcium carbonate, magnesium stearate, maltodextrin, and mixtures thereof. The various ingredients and the excipient and/or carrier are mixed and formed into the desired form using conventional techniques. The tablet or capsule of the present invention may be coated with an enteric coating that dissolves at a pH of about 6.0 to 7.0. A suitable enteric coating that dissolves in the small intestine but not in the stomach is cellulose acetate phthalate. Further details on techniques for formulation for and administration may be found in the latest edition of Remington's Pharmaceutical Sciences (Maack Publishing Co., Easton, Pa.).

In other embodiments, the dietary supplement is provided as a powder or liquid suitable for adding by the consumer to a food or beverage. For example, in some embodiments, the dietary supplement can be administered to an individual in the form of a powder, for instance to be used by mixing into a beverage, or by stirring into a semi-solid food such as a pudding, topping, sauce, puree, cooked cereal, or salad dressing, for instance, or by otherwise adding to a food e.g. enclosed in caps of food or beverage container for release immediately before consumption. The dietary supplement may comprise one or more inert ingredients, especially if it is desirable to limit the number of calories added to the diet by the dietary supplement. For example, the dietary supplement of the present invention may also contain optional ingredients including, for example, herbs, vitamins, minerals, enhancers, colorants, sweeteners, flavorants, inert ingredients, and the like.

In some embodiments, the dietary supplements further comprise vitamins and minerals including, but not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3; cyanocobalamin; sodium selenite; copper sulfate; vitamin A; vitamin C; inositol; potassium iodide. Suitable dosages for vitamins and minerals may be obtained, for example, by consulting the U.S. RDA guidelines.

In other embodiments, the present invention provides nutritional supplements (e.g., energy bars or meal replacement bars or beverages) comprising the composition according to the invention. The nutritional supplement may serve as meal or snack replacement and generally provide nutrient calories. Preferably, the nutritional supplements provide carbohydrates, proteins, and fats in balanced amounts. The nutritional supplement can further comprise carbohydrate, simple, medium chain length, or polysaccharides, or a combination thereof. A simple sugar can be chosen for desirable organoleptic properties. Uncooked cornstarch is one example of a complex carbohydrate. If it is desired that it should maintain its high molecular weight structure, it should be included only in food formulations or portions thereof which are not cooked or heat processed since the heat will break down the complex carbohydrate into simple carbohydrates, wherein simple carbohydrates are mono- or disaccharides. The nutritional supplement contains, in one embodiment, combinations of sources of carbohydrate of three levels of chain length (simple, medium and complex; e.g., sucrose, maltodextrins, and uncooked cornstarch).

Sources of protein to be incorporated into the nutritional supplement of the invention can be any suitable protein utilized in nutritional formulations and can include whey protein, whey protein concentrate, whey powder, egg, soy flour, soy milk soy protein, soy protein isolate, caseinate (e.g., sodium caseinate, sodium calcium caseinate, calcium caseinate, potassium caseinate), animal and vegetable protein and hydrolysates or mixtures thereof. When choosing a protein source, the biological value of the protein should be considered first, with the highest biological values being found in caseinate, whey, lactalbumin, egg albumin and whole egg proteins. In a preferred embodiment, the protein is a combination of whey protein concentrate and calcium caseinate. These proteins have high biological value; that is, they have a high proportion of the essential amino acids. See Modern Nutrition in Health and Disease, eighth edition, Lea & Febiger, publishers, 1986, especially Volume 1, pages 30-32. The nutritional supplement can also contain other ingredients, such as one or a combination of other vitamins, minerals, antioxidants, fiber and other dietary supplements (e.g., protein, amino acids, choline, lecithin, omega-3 fatty acids). Selection of one or several of these ingredients is a matter of formulation, design, consumer preference and end-user. The amounts of these ingredients added to the dietary supplements of this invention are readily known to the skilled artisan. Guidance to such amounts can be provided by the U.S. RDA doses for children and adults. Further vitamins and minerals that can be added include, but are not limited to, calcium phosphate or acetate, tribasic; potassium phosphate, dibasic; magnesium sulfate or oxide; salt (sodium chloride); potassium chloride or acetate; ascorbic acid; ferric orthophosphate; niacinamide; zinc sulfate or oxide; calcium pantothenate; copper gluconate; riboflavin; beta-carotene; pyridoxine hydrochloride; thiamin mononitrate; folic acid; biotin; chromium chloride or picolonate; potassium iodide; sodium selenate; sodium molybdate; phylloquinone; vitamin D3 ; cyanocobalamin; sodium selenite; copper sulfate; vitamin A; vitamin C; inositol; potassium iodide.

The nutritional supplement can be provided in a variety of forms, and by a variety of production methods. In a preferred embodiment, to manufacture a food bar, the liquid ingredients are cooked; the dry ingredients are added with the liquid ingredients in a mixer and mixed until the dough phase is reached; the dough is put into an extruder, and extruded; the extruded dough is cut into appropriate lengths; and the product is cooled. The bars may contain other nutrients and fillers to enhance taste, in addition to the ingredients specifically listed herein.

It is understood by those of skill in the art that other ingredients can be added to those described herein, for example, fillers, emulsifiers, preservatives, etc. for the processing or manufacture of a nutritional supplement.

Additionally, flavors, coloring agents, spices, nuts and the like may be incorporated into the nutraceutical composition. Flavorings can be in the form of flavored extracts, volatile oils, chocolate flavorings, peanut butter flavoring, cookie crumbs, crisp rice, vanilla or any commercially available flavoring. Examples of useful flavoring include, but are not limited to, pure anise extract, imitation banana extract, imitation cherry extract, chocolate extract, pure lemon extract, pure orange extract, pure peppermint extract, imitation pineapple extract, imitation rum extract, imitation strawberry extract, or pure vanilla extract; or volatile oils, such as balm oil, bay oil, bergamot oil, cedarwood oil, walnut oil, cherry oil, cinnamon oil, clove oil, or peppermint oil; peanut butter, chocolate flavoring, vanilla cookie crumb, butterscotch or toffee. In one embodiment, the dietary supplement contains cocoa or chocolate.

Emulsifiers may be added for stability of the nutraceutical compositions. Examples of suitable emulsifiers include, but are not limited to, lecithin (e.g., from egg or soy), and/or mono- and di-glycerides. Other emulsifiers are readily apparent to the skilled artisan and selection of suitable emulsifier(s) will depend, in part, upon the formulation and final product. Preservatives may also be added to the nutritional supplement to extend product shelf life. Preferably, preservatives such as potassium sorbate, sodium sorbate, potassium benzoate, sodium benzoate or calcium disodium EDTA are used.

In addition to the carbohydrates described above, the nutraceutical composition can contain natural or artificial (preferably low calorie) sweeteners, e.g., saccharides, cyclamates, aspartamine, aspartame, acesulfame K, and/or sorbitol. Such artificial sweeteners can be desirable if the nutritional supplement is intended to be consumed by an overweight or obese individual, or an individual with type II diabetes who is prone to hyperglycemia.

Moreover, a multi-vitamin and mineral supplement may be added to the nutraceutical compositions of the present invention to obtain an adequate amount of an essential nutrient, which is missing in some diets. The multi-vitamin and mineral supplement may also be useful for disease prevention and protection against nutritional losses and deficiencies due to lifestyle patterns.

The dosage and ratios of hydroxytyrosol administered via a nutraceutical composition will, of course, vary depending upon known factors, such as the physiological characteristics of the particular composition; the age, health and weight of the recipient; the nature and extent of the symptoms; the kind of concurrent treatment; the frequency of treatment; and the effect desired which can be determined by the expert in the field with normal trials, or with the usual considerations regarding the formulation of a nutraceutical composition.

A food or beverage suitably contains about 0.5 mg to about 1000 mg of hydroxytyrosol per serving. If the composition is a pharmaceutical composition such a composition may contain hydroxytyrosol in an amount from about 1 mg to about 2000 mg per dosage unit, e.g., per capsule or tablet, or from about 1 mg per daily dose to about 3000 mg per daily dose of a liquid formulation.

The present invention is also directed to pharmaceutical compositions comprising hydroxytyrosol for treating or preventing age-related macular degeneration in humans, for maintaining the eye health in animals (preferably in humans), especially in elderly animals (preferably elderly humans), for improving the vision of animals (preferably of humans), for maintaining the high resolution vision in animals (preferably in humans) and/or for maintaining the visual acuity in animals (preferably in humans), as well as for maintaining the visual performance in animals (preferably in humans) and/or the visual function in animals (preferably in humans).

The pharmaceutical compositions according to the invention preferably further comprise a pharmaceutically acceptable carriers. Suitable pharmaceutical carriers are e.g. described in Remington's Pharmaceutical Sciences, supra, a standard reference text in this field. Examples of such pharmaceutically acceptable carriers are both inorganic and organic carrier materials, suitable for oral administration and include water, gelatin, gum arabic, lactose, starch, magnesium stearate, talc, vegetable oils, and the like.

The pharmaceutical composition may further comprise conventional pharmaceutical additives and adjuvants, excipients or diluents, including, but not limited to, water, gelatin of any origin, vegetable gums, ligninsulfonate, talc, sugars, starch, gum arabic, vegetable oils, polyalkylene glycols, flavoring agents, preservatives, stabilizers, emulsifying agents, buffers, lubricants, colorants, wetting agents, fillers, and the like.

The dosages and ratios of the individual components in a pharmaceutical composition can be determined by the expert in the field with normal preclinical and clinical trials, or with the usual considerations regarding the formulation of pharmaceutical composition.

In a preferred embodiment hydroxytyrosol is administered via a pharmaceutical composition either in the form of a single dose or by multiple doses in an amount of at least 0.3 mg/ kg bodyweight/day, preferably in an amount of 1-450 mg/kg body weight/day, most preferably in an amount of 4-140 mg/kg body weight/day.

The compositions according to the present invention may be in any galenic form that is suitable for administering orally to the animal body including the human body, e.g. in solid form, for example as (additives/supplements for) food or feed, food or feed premixes, fortified food or feed, tablets, pills, granules, dragées, capsules, and effervescent formulations such as powders and tablets, or in liquid form, for instance in the form of solutions, emulsions or suspensions, for example as beverages, pastes and oily suspensions. The pastes may be filled into hard or soft shell capsules, whereby the capsules feature e.g. a matrix of (fish, swine, poultry, cow) gelatin, plant proteins or ligninsulfonate. The nutraceutical and pharmaceutical compositions may be in the form of controlled (delayed) release formulations.

The invention is now further illustrated by the following, non-limiting examples.

### EXAMPLES

### Example 1

### Soft Gelatin Capsule

Soft gelatin capsules are prepared by conventional procedures providing a dose of hydroxytyrosol of 50 mg per capsule. A suitable daily dose is 1 to 5 capsules.

Other ingredients: glycerol. Water, gelatine, vegetable oil

### Example 2

### Hard gelatin Capsule

Hard gelatin capsules are prepared by conventional procedures providing a dose of hydroxytyrosol of 75 mg per capsule. A suitable daily dose is 1 to 5 capsules.

Other ingredients:

Fillers: lactose or cellulose or cellulose derivatives q.s.

Lubricant: magnesium stearate if necessary (0.5%)

### Example 3

### Tablet

Tablets are prepared by conventional procedures providing as active ingredient 100 mg of hydroxytyrosol per tablet, and as excipients microcrystalline cellulose, silicone dioxide (SiO₂), magnesium stearate, crosscarmellose sodium ad 500 mg.

### Example 4

### Soft Drink

A soft drink containing hydroxytyrosol may be prepared as follows:

| | |
|---|---|
| ingredient | [g] |

A. juice concentrates and water soluble flavours

| | |
|---|---|
| 60.3° Brix, 5.15% acidity | 657.99 |
| 43.5° Brix, 32.7% acidity | 95.96 |
| Orange flavour, water soluble | 3.43 |
| Apricot flavour, water soluble | 6.71 |
| water | 26.46 |

B. color

| | |
|---|---|
| β-carotene 10% CWS | 0.89 |
| water | 67.65 |

C. Acid and antioxidant

| | |
|---|---|
| Ascorbic acid | 4.11 |
| Citric acid anhydrous | 0.69 |
| water | 43.18 |

D. stabilizers

| | |
|---|---|
| pectin | 0.20 |
| Sodium benzoate | 2.74 |
| water | 65.60 |

E. oil soluble flavours

| | |
|---|---|
| Orange flavour, oil soluble | 0.34 |
| Orange oil distilled | 0.34 |

F. active ingredient

| | |
|---|---|
| Hydroxytyrosol | Amount providing 15 mg |

Fruit juice concentrates and water soluble flavours are mixed without incorporation of air. The color is dissolved in deionized water. Ascorbic acid and citric acid are dissolved in water. Sodium benzoate is dissolved in water. The pectin is added under stirring and dissolved while boiling. The solution is cooled down. Orange oil and oil soluble flavours are premixed. The active ingredient as mentioned under F is stirred into the fruit juice concentrate mixture of A.

In order to prepare the soft drinks all components A-F are mixed together before homogenizing using a Turrax and then a high-pressure homogenizer (p₁=200 bar, p₂=50 bar).

### Example 5

### Cell Culture test with Human ARPE-19 Cells

The human ARPE-19 cells (a human retinal pigment epithelial cell-line) were maintained in DMEM-F12 medium (Dulbecco's modified Eagle's medium) supplemented with 10% fetal bovine serum, 0.348% sodium bicarbonate, 2 mM L-glutamine, 100 U/mL penicillin and 100 μg/ml streptomycin. Cell cultures were maintained at 37° C. in a humidified atmosphere of 95% air and 5% CO₂. The medium was changed every 3 to 4 days. ARPE-19 cells were used within 10 generations.

### Reagents

Acrolein was purchased from Sinopharm Chemical Reagent Co., Ltd (Shanghai, China). Unless otherwise stated, all reagents were purchased from Sigma-Aldrich Chemical Co. (St. Louis, Mo.). Hydroxytyrosol was synthesized chemically.

### Acrolein Exposure and HTS Supplementation

All experiments were performed with an 80% confluence monolayer grown in 96-well plates or 6 well plates. Hydroxytyrosol (HTS) was dissolved in DMSO (dimethyl sulfoxide). Acrolein was dissolved in PBS (phosphate buffer salt) each time immediately before an experiment. For the acute toxicity study, cells were exposed to acrolein for 24 hours. The protective effects of HTS (Hydroxytyrosol) were studied with the acute toxicity model by pre-treating cells with HTS for 48 hours or for 7 days.

### MTT Assay for Cell Viability

The MTT assay reduction assay was used as a qualitative index of cell viability. The optical densities were read at 555 nm using a microplate spectrophotometer (Spectra Max 340, Molecular Dabices, Sunnyvale, Calif.). Absorbance values were normalized with untreated cells to calculate the changes in cell viability.

### JC-1 Assay for Mitochondrial Membrane Potential

Mitochondrial potential change (ΔΨ) was assessed in live APRE-19 cells using the lipophilic cationic probe 5,5′,6,6′-tetrachloro-1,1′,3,3′-tetraethylbenzimidazol-carbocyanine iodine (JC-1). For quantitative fluorescence measurement, cells were rinsed once after JC-1 staining and scanned with a multi-label counter (Wallac 1420; PerkinElmer Life Sciences, Wellesley, Mass.) at 485 nm excitation and 535 nm and 590 nm emission, to measure green and red JC-1 fluorescence respectively. Each well was scanned at 25 areas rectangularly arranged in 5×5 pattern with 1 mm intervals and approximate beam area of 1 mm² (bottom scanning). For microscopic observation of JC-1 staining ARPE-19, images were collected with FITC and TRITC fluorescence filter cubes on a microscope (Axiover25; Carl Zeiss Meditec, Inc., Thornwood, N.Y.) equipped with a charge-coupled device (CCD) digital camera (Diagnostic Instruments, Sterling Heights, Mich.), and processed with image-management software (Photoshop ver. 7.0; Adobe Systems, Mountain View, Calif.).

### Total Antioxidant Power

Intracellular total antioxidant power of ARPE-19 cells was assayed by a commercially available assay kit (Total antioxidant power, A-015, Jiancheng Biochemical Inc., Nanjing, China) according to the kit instructions.

### Superoxide Dismutase (SOD) Measurement

Intracellular SOD activity was measured by Superoxide Dismutase Assay Kit (A001, Jiancheng Biochemical Inc., Nanjing, China) according to the kit instructions.

### Assay for GSH Levels

The GSH level was assayed with a commercially available assay kit (Jiancheng Biochemical Inc., Nanjing, China) using an assay based on a thiol-specific reagent, dithionitrobenzoic acid (DTNB), and the adduct was measured spectrophotometrically at 412 nm.

### Detection of Protein Carbonyls

For determination of protein carbonyls, a measure of protein oxidation, cells were grown on 100 mm plates. Protein carbonyls in soluble proteins were assayed with the Oxyblot protein oxidation detection kit (Cell BioHTSbs, San Diego, Calif.).

Total levels of Nuclear Factor-E2-reHTSted Factor 2 (Nrf2)

Cells were grown on 100 mm plates and were homogenized (1:10) in RIPA Buffer [150 mM PBS containing 1% (vol/vol) Igepal CA630, 0.5% (wt/vol) sodium deoxycholate, 0.1% (wt/vol) SDS, and 5 μ/μl protease inhibitor mixture], pH 7.4, and 50 μg of protein was used for Western analysis of total Nrf2 levels and probed with anti-Nrf2 antibodies (Santa Cruz) at a 1:500 titer. Chemiluminescent detection was done by an ECL Western Blotting Detection kit from Amersham Pharmacia.

### Intracellular Calcium Assay

Intracellular Ca⁺⁺ levels were determined by a commercially available assay kit (C004, Jiancheng Biochemical Inc., Nanjing, China) according to the kit instructions.

### Assays for Activities of Mitochondrial Complex I, II, and III

ARPE-19 cells were cultured in 100 mm plates, washed in PBS, resuspended in an appropriate isotonic buffer (0.25 M sucrose, 5 mM Tris-HCl, pH 7.5, and 0.1 mM phenylmethylsulfonyl fluoride), and homogenized. Mitochondria were isolated by differential centrifugation of the cell homogenates. NADH-CoQ oxidoreductase (Complex I), succinate-CoQ oxidoreductase (complex II), CoQ-cytochrome c reductase (complex III) were assayed spectrometrically using the conventional assays with minor modifications.

### Statistical Analysis

Data were presented as mean±SD of two or three separate experiments, as specified in the figure legends. Statistical significance was calculated using Prism software (version 4.0a) using one-way ANOVA, and p value<0.05 was considered significant.

### Results

In the Figures the following abbreviations are used:
“C”=control;
“A”=acrolein;
“H”=hydroxytyrosol;
“H+A”=hydroxytyrosol+acrolein;
“C+H”=control+hydroxytyrosol;
“HTS×−A”=hydroxytyrosol in different concentrations with acrolein.
   Protective effect of HTS on acrolein-induced decrease in cell viability in ARPE-19 cells

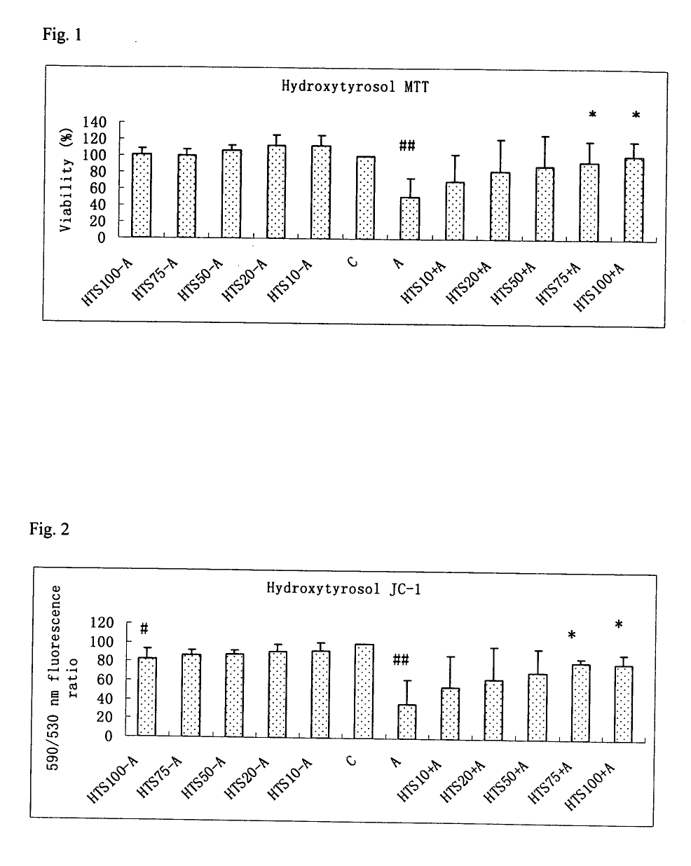

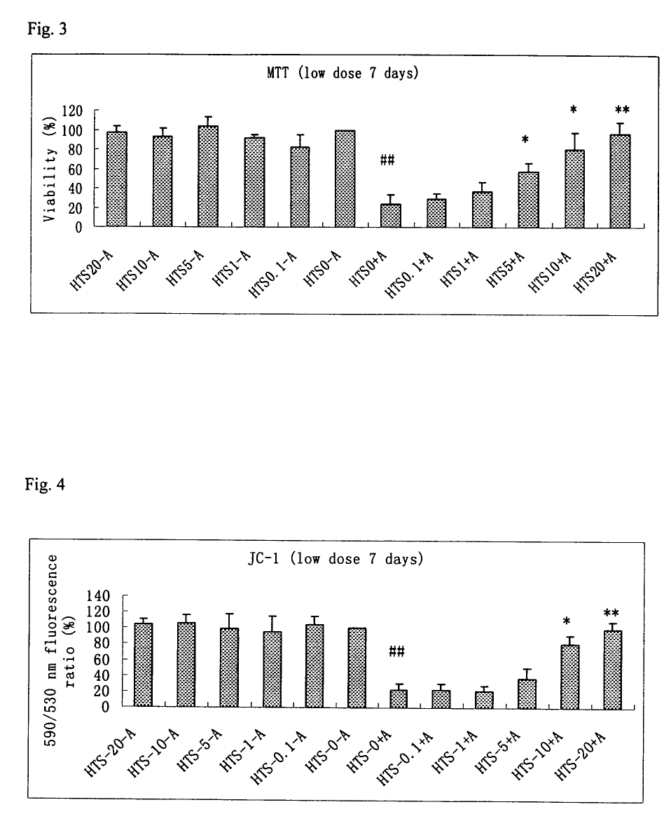

The ARPE-19 cells were seeded at 4×10⁴ per well in a 96 well plate. Cells were pretreated with different levels of HTS for 48 hours when cells were 80% confluent and then treated with 75 μM acrolein for 24 hours. HTS itself had no apparent effect on cell viability in the concentrations used (10-100 μM HTS in ARPE-19) (FIG. 1). The pretreatments of ARPE-19 cells with HTS resulted in a significant protection against 75 μM acrolein-induced toxicity. In the 10-20 μM range, HTS could protect against an acute acrolein-induced decrease in cell viability. HTS at 20 μM completely abolished acrolein toxicity when ARPE-19 cells were pretreated for 7 days (FIG. 3).

FIG. 1 shows the protective effects of HTS on acrolein-induced decrease in cell viability measured by the MTT assay. ARPE-19 cells with 48 h-HTS pretreatment. Values are mean±SD of data from four separate experiments; each experiment was performed in triplicate. ##P<0.01 vs. control (HTS 0 μM). *P<0.05 vs. acrolein 75 μM without HTS.

FIG. 3 shows the protective effects of HTS on acrolein-induced decrease in cell viability measured by the MTT assay. ARPE-19 cells with 7 days-HTS pretreatment. Values are mean±SD of data from four separate experiments, each experiment performed in triplicate. ##P<0.01 vs. HTS 0. *P<0.05 and **p<0.01 vs. acrolein without HTS.

### Protective Effect of HTS on Acrolein-Induced Decrease in Mitochondrial Membrane Potential in ARPE-19 Cells

Similar to the results on cell viability, HTS itself had no apparent effect on mitochondrial membrane potential in both ARPE-19 cells in the concentrations used (10-100 μM HTS in ARPE-19 cells). Similar to the protection on cell viability, a 7-day long pretreatment enhanced the protective effect of HTS. As shown in FIGS. 2, 4 and 10 and HTS significantly protected the acute acrolein-induced decrease in mitochondrial membrane potential. HTS at concentrations lower than 10 μM showed no protective effect against acrolein-induced cell toxicity in ARPE-19.

FIG. 2 shows the protective effects of HTS on acrolein-induced decrease mitochondrial membrane potential measured by JC-1 assay. ARPE-19 cells with 48 h-HTS pretreatment. Values are mean±SD of data from three separate experiments; each experiment was performed in triplicate. #p<0.05 and ##P<0.01 vs. HTS 0. *P<0.05 vs. HTS 0+acrolein 75 μM.

FIG. 4 shows the protective effects of HTS on acrolein-induced decrease mitochondrial membrane potential measured by JC-1 assay. ARPE-19 cells with 7 days-HTS pre-treatment. Values are mean±SD of data from one representative of three experiments, performed in triplicate. ##p<0.01 vs. control *p<0.05 and **p<0.01 vs. acrolein without HTS.

### HTS Modulates the Acrolein-Induced Decrease in Intracellular SOD in ARPE Cells

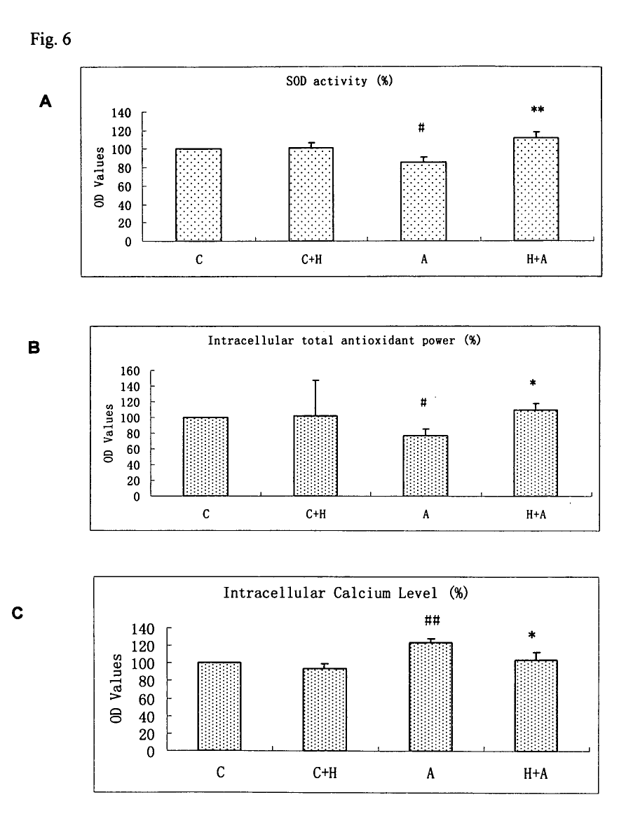

Treatment with 75 μM acrolein caused a significant decrease in intracellular SOD activity in ARPE-19 cells (FIG. 6A). HTS pretreatment at 100 μM prevented the decrease in SOD activity (FIG. 6A). Treatment with 100 μM HTS without acrolein increased intracellular SOD activity in untreated normal ARPE-19 cells (FIG. 6A).

### HTS Modulates the Acrolein-Induced Decrease in Intracellular Total Antioxidant Power in ARPE-19 Cells

Acrolein at 75 μM decreased intracellular antioxidant power in ARPE-19 cells. Pretreatment with 100 μM HTS prevented the cells from acrolein-induced decrease (FIG. 6B). Again as on the SOD activity, this protection may be due to the antioxidant activity of HTS itself since HTS at 100 μM without acrolein elevated the intracellular total antioxidant power (FIG. 6B).

FIG. 6 shows the acute acrolein exposure (24 hour)-induced changes in intracellular SOD, antioxidant power, and Ca²⁺ levels and modulation by HTS (48 hour-pretreatment) in ARPE-19 cells. Values are mean±SD of data from 3 separate experiments and each experiment was performed in triplicate. #p<0.05 and ##p<0.01 vs. control, *p<0.05 and **p<0.01vs. 75 μM acrolein without HTS.

### HTS Modulates the Intracellular Ca2+ Increase Caused by Acrolein in ARPE-19 Cells

Mitochondrial dysfunction usually results in an increase in cytoplasmic Ca²⁺ level, which is a biomarker of oxidative stress and mitochondrial dysfunction. Treatment of ARPE-19 cells with 75 μM acrolein caused a significant increase in intracellular Ca²⁺ level (FIG. 6C). Pretreatment with 100 μM HTS before 75 μM acrolein significantly inhibited the increase in Ca²⁺. HTS at 100 μM without acrolein did not significantly change the intracellular Ca²⁺ level in ARPE-19 cells.

### HTS Inhibited Acrolein-Induced Decreases in GSH Level in ARPE-19 Cells

Pretreatment ARPE-19 cells with HTS for 48 hours showed a trend in increasing GSH level (FIG. 7). Acrolein at 75 μM for 24 hours caused a significant decrease in the GSH level and HTS at 100 μM for 48 hours provided a full protection to GSH level (FIG. 7).

FIG. 7 shows the acrolein-induced changes in GSH levels and protective effect of HTS in ARPE cells. HTS pretreatment for 48 hour and acrolein exposure for 24 hours. Values are mean±SD of data from four separate experiments and each experiment was performed in triplicate ##p<0.01 vs. control, *p<0.05 vs. 75 μM acrolein without HTS.

### HTS Inhibited Acrolein-Induced Increase in Protein Carbonyls in ARPE-19 Cells

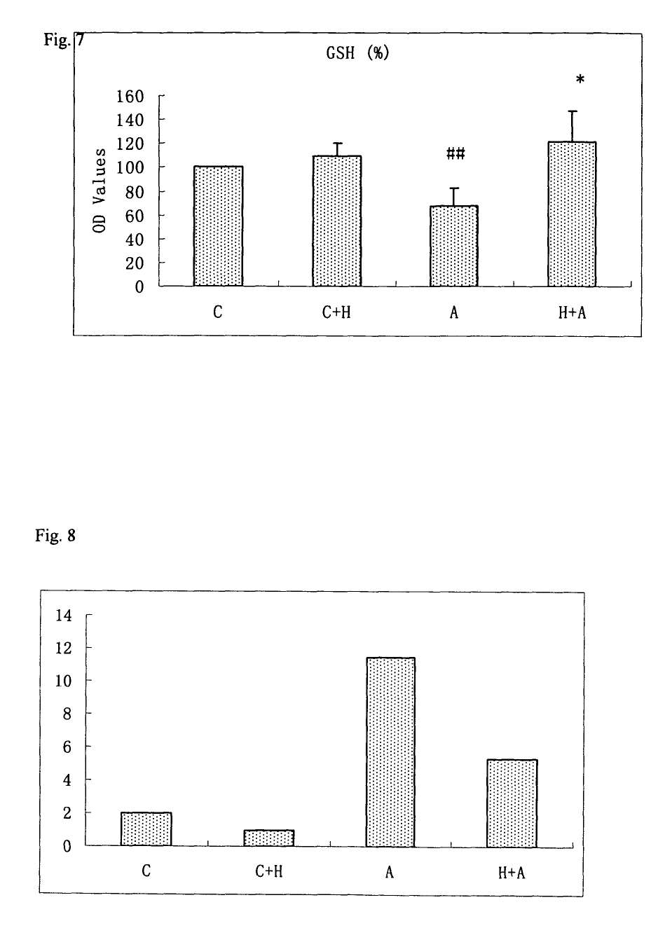

Acrolein at 75 μM for 24 hours caused a significant increase in protein carbonyls, an index of protein oxidation (FIG. 8). Pretreatment with 100 μM HTS for 48 hours showed significant inhibition on acrolein-induced increase in protein carbonyls (FIG. 8).

FIG. 8 shows the acrolein-induced changes in protein carbonyls and protective effect of HTS in ARPE cells assayed by western blotting. HTS pretreatment for 48 h and acrolein exposure for 24 h. Representative quantitative data of protein carbonyls from 4 separate similar experiments.

### HTS Modulates Acrolein-Induced Decrease in Total Nrf2 Expression in ARPE-19 Cells

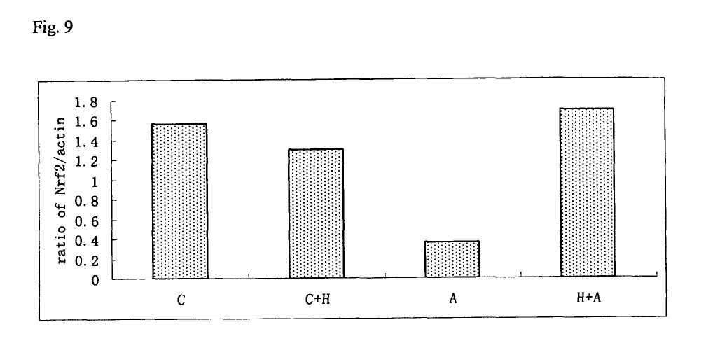

Acrolein at 75 μM for 24 h caused a significant decrease in both total Nrf2 expression in ARPE-19 cells, and pretreatment with HTS at 100 uM for 48 h significantly prevented the cells from acrolein-induced decrease in total Nrf2 (FIG. 9).

FIG. 9 shows the acrolein-induced changes in total Nrf2 expressions and protective effect of HTS in ARPE cells assayed by western blotting. HTS pretreatment for 48 hours and acrolein exposure for 24 hours.

### HTS Modulates Acrolein Induced Decreases of Mitochondrial Complex I, II, and III Activity in ARPE-19 Cells

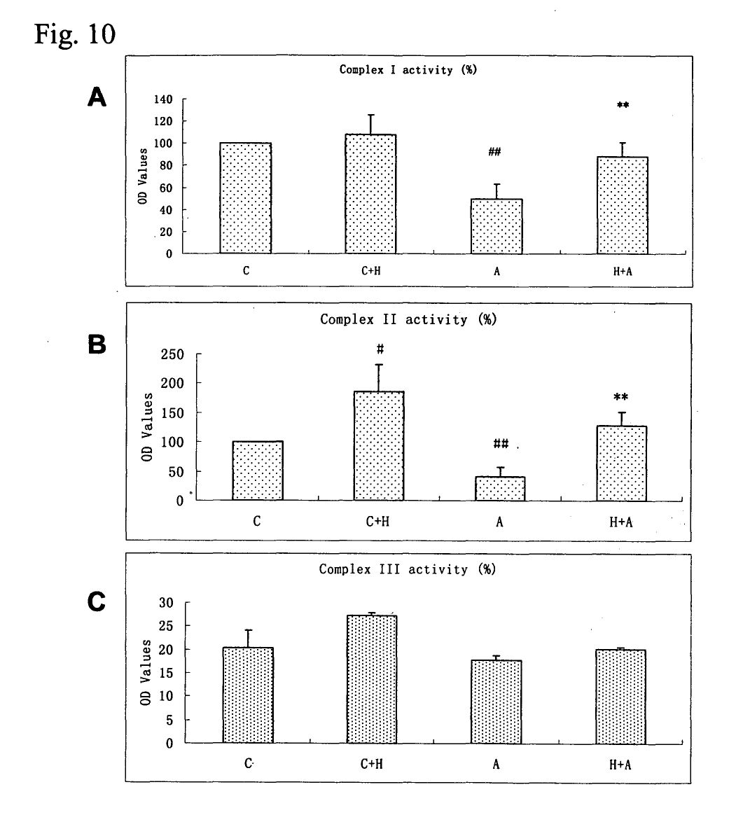

Acrolein at 75 μM for 24 h caused a significant decrease in the activity of mitochondrial complex I, II, and III in ARPE-19 cells 1 (FIGS. 10A, B, and C). Pretreatment with 100 μM HTS showed significant protections on complex I (FIG. 10A), complex II (FIG. 10B), and complex III (FIG. 10C).

FIG. 10 shows the protection by HTS of the acrolein-induced decrease in mitochondrial complexes in ARPE-19 cells. (A) Complex I, (B) Complex II, and (C) Complex III. ARPE-19 cells were pretreated with different concentrations of HTS and then treated with 75 μM acrolein. Values are mean±SD of data from four separate experiments for complex I, and three separate experiments for complex II and III, and each experiment was performed in duplicate. #p<0.05 and ##p<0.01 vs. control, **p<0.01 vs. 75 μM acrolein without HTS.

1.-27. (canceled)

28. Method for treating or preventing age-related macular degeneration in humans by administering a composition comprising hydroxytyrosol in an effective amount to said humans.

29. The method according to claim 28 wherein the humans are elderly.

30. The method according to claim 28 wherein the hydroxytyrosol is present in an olive extract.

31. The method according to claim 28 wherein the hydroxytyrosol is present in a food or nutraceutical.

32. Method for maintaining the eye health in animals, for improving the vision of animals, for maintaining the high resolution vision in animals, for maintaining the visual acuity in animals, for maintaining the visual performance in animals and/or for maintaining the visual function in animals by administering (a composition comprising) hydroxytyrosol in an effective amount to said animals.

33. The method according to claim 32, wherein the animals are humans.

34. The method according to claim 32 wherein the humans are elderly.

35. The method according to claim 32 wherein the hydroxytyrosol is present in an olive extract.

36. The method according to claim 32 wherein the hydroxytyrosol is present in a food or nutraceutical.
